(19) United States
(12) Patent Application Publication
(10) Pub. No.: US 2005/0148545 A1
(43) Pub. Date: Jul. 7, 2005

(54) Glucosamine organic acid adducts

(76) Inventors: Lawrence E Fosdick, Oskaloosa, IA (US); Timothy W Bauer, Naperville, IL (US); John A Bohlmann, Ottumwa, IA (US); Ki-Oh Hwang, Cedar Rapids, IA (US); Brent D Rogers, Ottumwa, IA (US)

Correspondence Address: CARGILL, INCORPORATED, LAW DEPARTMENT, P. O. BOX 5624, MINNEAPOLIS, MN, 55440-5624, US

(21) Appl. No.: 10/497,943

(22) PCT Filed: Dec. 6, 2002
(86) PCT No.: PCT/US02/39309

Related U.S. Application Data
(60) Provisional application No. 60/339,004, filed on Dec. 7, 2001.

(51) Int. Cl. A61K 31/7008; C07H 5/06; A23G 3/00

(52) U.S. Cl. 514/62; 536/18.7; 426/658

(57) ABSTRACT

The invention pertains to compositions containing glucosamine and organic acids, methods of making such compositions and methods of using such compositions.

### CROSS REFERENCE TO RELATED APPLICATIONS

This application claims priority from U.S. provisional application No. 60/339,004 filed Dec. 7, 2001, which is herein incorporated by reference.

### FIELD

The invention provides compositions containing glucosamine organic acid adducts and methods of making and using the same.

### BACKGROUND

Presently the majority of the glucosamine available for use as a dietary supplement is in the form of the salt glucosamine hydrochloride or the salt glucosamine sulfate. Formulators add glucosamine as an individual ingredient to a multi-component formulation, such as tablets, or dietary supplements such as supplement bars or supplement beverages. Hence, many production processes that incorporate glucosamine into final products have separate vessels (hoppers) that allow glucosamine to be individually added.

In order to increase efficiency of production (i.e. reduce the number of vessels) formulators sometimes use dry blends of ingredients that are then added during the production process from a single vessel. Dry blends are mixtures of two distinct ingredients that act independently of each other. Unfortunately, containers of dry blends do not maintain homogeneity and therefore, the ingredients segregate to different parts of the container due to the different physical characteristics of the individual ingredients. Glucosamine hydrochloride and glucosamine sulfate are also known to have a bitter taste, which taste can lead consumers to search for better tasting alternatives.

Therefore, a need exists for a bulk glucosamine composition that can be taken as a dietary supplement, used to formulate complex dietary supplements, and/or used as a food ingredient, and which has a better taste profile.

### SUMMARY

Glucosamine organic acid adducts (GOA) are disclosed. The disclosed adducts mainly contain, glucosamine, one or more organic acids or salts thereof, moisture, one or more balancing ions, and one or more inorganic salts. For example the GOA may contain glucosamine and 1, 2, 3, 4, or 5 different organic acids, which together form crystals with glucosamine. Certain embodiments of the composition typically have a substantially uniform or homogenous concentration of glucosamine and organic acid throughout. The uniformity can be obtained, for example, by crystallizing or surface crystallizing the glucosamine and one or more organic acids. The GOAs described herein can be used in a variety of applications, such as food ingredients and/or dietary supplements. The GOAs are useful for, among other things, tableting, especially for making chewable tablets, and altering the taste profiles of glucosamine containing compositions.

Certain embodiments of the GOA compositions are substantially homogeneous. The GOA compositions can be combined with other materials, including for example crystalline materials, and/or may be used as an ingredient in food products or nutritional supplements. The GOA compositions can be also used in tablet formulations.

The ratio of glucosamine to organic acids in the GOA embodiments can vary. For example, the glucosamine concentration can be greater than, or equal to, the total organic acid concentration. In alternative embodiments, the total organic acid concentration is greater than the glucosamine concentration. Exemplary embodiments of GOA compositions can contain glucosamine to total organic acid concentration (G:OA) ratios such as 100:1, 75:1, 50:1, 25:1, 10:1, 5:1, 1:1, 1:5, 1:10, 1:25, 1:50, 1:75, or 1:100. Alternatively, ranges between these ratios, such as from about 100 to about 75:1, or from about 75 to about 50:1 can be used. The moisture content of the GOA can also vary. For example, the moisture content can be less than about 20 weight percent of the GOA or less than 15, 10, 5, 3, 2 or 1 weight percent of the GOA. However, other moisture concentrations are also useful.

Methods for producing GOA are also disclosed. As known to persons of ordinary skill in the art, once the GOAs are disclosed, various methods can be used to produce such GOA. Certain example methods involve adding one or more organic acids to a solution containing glucosamine, removing water from the solution until crystals form and collecting the crystals, wherein the crystals contain the glucosamine organic acid adduct. Other methods provided by the invention involve adding a base, such as NaOH, KOH, or the salt form of an organic acid to the solution containing glucosamine and/or organic acids.

### BRIEF DESCRIPTION OF THE DRAWINGS

FIG. 1 is a spectrum from a FTIR (Fourier Transform Infrared) Spectrometry analysis of an embodiment of the glucosamine-citrate adduct as made by the process disclosed in Example 1.

FIG. 2 is an FTIR spectrum of a prior art glucosamine hydrochloride (Lot 27126A), CAS No. 66-84-2 (Phanstiehl Laboratories, Inc., Waukegan, Ill.) used as a control.

FIG. 3 is an FTIR spectrum of citric acid produced at Cargill, Inc., Eddyville, Iowa, CAS No. 77-92-9 used as a control.

### I. DETAILED DESCRIPTION

A. Process for Obtaining Glucosamine

Glucosamine for use in the embodiments of the GOAs may be from any suitable source, such as bacterial biomass, or chitin containing sources such as fungal biomass or shellfish. Free glucosamine can be produced by a variety of methods and from various sources as is well known to those of ordinary skill in the art. For example, free glucosamine can be produced by treating glucosamine hydrochloride (or sulfate) in a solution either with an inorganic base, for example, LiOH, NaOH, KOH, CaO, and/or Ca(OH)₂, or an organic base such as sodium citrate. The amount of base used depends on the glucosamine source and the chosen base(s). For example, from about 0 to about 1.1 equivalents of base to glucosamine may be used. Alternatively, the amount of base used may be determined from the quantity of organic acid that is to be used to make the GOA, for example see the embodiment disclosed in Example 2. The pH of the solution may be kept below about 11, to avoid oxidation and/or color formation. Other pH levels may also be employed.

A base can be added to the solution containing glucosamine in various manners as is known to those of ordinary skill in the art. For example, the base may be added as a solid, slurry, and/or solution. Adding the base as a solution or as a slurry may avoid extreme localized heat fluctuations and/or localized reactions. Maintaining the temperature of the solution containing glucosamine at about 37° C. or less may help stabilize free glucosamine.

Glucosamine can be isolated from the solution containing glucosamine described above using various methods known to those skilled in the art, such as precipitation, extraction, or chromatographic methods. However, in the experiments detailed below free glucosamine in solution was used. The free glucosamine in basic solution may be unstable, and the compound may oxidize or decompose easily in solution developing color at a relatively high pH.

B. Making Glucosamine-Organic Acid Adduct (GOA)

The glucosamine organic acid adduct (GOA) can be obtained for example, by adding an organic acid to a glucosamine hydrochloride solution, a glucosamine sulfate solution, or a free glucosamine solution. Organic acids such as acetic acid, ascorbic acid, formic acid, lactic acid, maleic acid, malic acid, propionic acid, succinic acid, fumaric acid, citric acid, nicotinic acid, or combinations thereof may be used. Addition of acid can be done at ambient temperature (25° C.-37° C.), or at lower temperatures.

Forming certain embodiments of GOA may be accomplished using mono-, di-, and/or polyprotic acids. The amount of acid used may be, for example, from about 0.2 molar ratio to about 5 molar ratio to glucosamine, but more or less acid may be desirable for specific applications. Addition of organic acid can be done either in solution or with the help of an ionic exchange chromatographic system, for example an ion exchange resin loaded with the conjugate base of the organic acid. If it is desired, de-salting may be done, for example, through membrane filtration, dialysis, or re-crystallization.

After enough acids are added to the mixture, the glucosamine is stable enough to be treated at elevated temperatures. The mixture can then be stirred for between about 0.5 to about 24 hours, and/or kept at elevated temperature to facilitate the formation of the GOA.

The resulting mixture containing glucosamine and organic acid can then be evaporated by any suitable means to concentrate the solution and allow crystals to form. Evaporation is generally done under vacuum between about 5 and about 26 inches of mercury and at elevated temperature (between about 35° C. to about 60° C.) to evaporate the water. The ratio of organic acid to glucosamine can be varied depending on the evaporation process. Virtually all of the water is removed to produce wet crystals, the ratio of acid to glucosamine will be the same as the starting mixture.

In another embodiment, the evaporation can be stopped after crystals form but before the water is completely removed. In this case, the ratio of glucosamine to acid will depend on the relative solubilities of the glucosamine and the organic acid. The final product composition can be controlled by balancing the starting concentration of the organic acid and glucosamine, the solubilities of the organic acid and the glucosamine, and the extent of evaporation.

In yet another embodiment, a water-miscible solvent, such as methanol, ethanol, or isopropanol, is used. The organic solvent decreases the solubilities of the organic acid and glucosamine, and provides for control of the product composition.

The mixture containing organic acid and glucosamine can stand for a period of time at the desired temperature to control the crystallization process. This is a process commonly referred to as digestion, which results in larger crystals. The crystallization process is an equilibrium process, and smaller crystals, having larger surface areas, tend to dissolve more rapidly than larger crystals. The dissolved components tend to crystallize onto the larger crystals.

Crystals obtained through crystallization can be separated using any suitable separation method, for example, decantation, filtration or centrifugation.

C. Characterization of the Glucosamine-Organic Acid Adduct (GOA)

1. Description of GOA

A glucosamine organic acid (GOA) adduct as described herein is a dry, crystalline adduct that contains primarily glucosamine and one or more organic acids, such as citric acid, propionic acid, acetic acid, ascorbic acid, lactic acid, amino acid such as glutamic acid, or other organic acids. As used herein adduct refers to a complex in which the glucosamine is bound with an organic acid without significantly changing the chemical character of either the glucosamine or the organic acid. For example components of the adducts are non-covalently bonded through either dispersive or non-dispersive bonding, such as ionic bonds, Van der Waal interactions, and/or hydrogen bonding. Therefore, a GOA is distinct from dry blends that contain pure glucosamine crystals and pure organic acid crystals.

Dry blends are mixtures that contain dry glucosamine hydrochloride or dry glucosamine sulfate and dry organic acid. Generally, dry blends are prepared by adding dry forms of glucosamine and organic acid together and mixing. Therefore, dry blends contain mixtures of glucosamine crystals and organic acid crystals that can be separated, however, this is not the case for GOA.

The GOA crystals are distinct from pure glucosamine hydrochloride crystals (which are rhomboid or bipyramidal in shape), and pure organic acid crystals such as citrate crystals (which are rounded needle shape with a translucent appearance). Some embodiments of GOA crystals with high glucosamine content show only some pyramidal characteristics. Similarly, some embodiments of GOA crystals containing high concentrations of citrate are slightly translucent, but clearly distinguishable from pure citrate crystals.

As mentioned above, in addition to glucosamine and organic acid, some embodiments of the GOA can contain balancing ions that provide charge neutrality in the adduct. These ions can be cations, such as lithium, sodium or potassium, and/or anions such as chloride, bromide, sulfate, or organic anions, such as the conjugate base of the organic acid used to prepare the adduct.

Another method of differentiating between GOA and dry blended glucosamine organic acid mixtures is by testing for homogeneity. Homogeneity as used herein describes that a container of GOA contains the same proportion of the components of the GOA throughout the container regardless of particle size. In other words, a sample taken from the top of a container of GOA is substantially similar to a sample taken from the bottom of the same container. Dry blends of glucosamine hydrochloride and citrate fail to form stable homogeneous mixtures because the two components segregate due to differences in crystal size, density and shape.

Another method of determining that a GOA sample contains a homogeneous crystalline material is by using standard light microscopy. One of ordinary skill in the art will appreciate that test samples can be taken from different sections of a large sample or container of GOA. These test samples can be viewed under a light microscope. The crystals in the test samples will look substantially the same and, therefore, the large sample is deemed homogeneous. This is in contrast to what would be seen if a dry blend was viewed. Test samples taken from different portions of a dry blend would show that the proportion of glucosamine crystals to the organic acid crystals is different between test samples.

Homogeneity of a GOA preparation can be expressed statistically with respect to the standard deviations of the method(s) used to analyze the samples. A preparation of an embodiment of a GOA is considered homogeneous if the differences between multiple samples from a single container vary by no more than about 130% to about 110% of the analytical confidence limit. Homogeneity can be described as “substantial” when the samples from a single container vary by no more than 130% of the analytical confidence limit. Homogeneity can be described as “significant” when the samples from a single container vary by no more than 120% of the analytical confidence limit. Homogeneity can be described as “high” when the samples from a single container vary by no more than 110% of the analytical confidence limit. The confidence limit for an analytical method is a well-known statistical figure of merit (Chemical Separations and Measurements, D. G. Peters, J. M. Hayes, and G. M. Hieftie, 1974 W. B. Saunders company, Philadelphia, Pa. ISBN 0-7216-7203-5, chapter 2). Values differing by quantities less than or equal to the confidence limit are considered statistically equivalent. The confidence limit for a method is calculated from the method variance using a Student t-table, and the general formula CL=±t_{α,φS}, where α is the uncertainty, φ is the number of degrees of freedom (number of measurements −2) and S is the standard deviation of the series of measurements.

Homogeneity of GOAs compared to dry-blended mixtures can be determined by proximate analysis to determine the glucosamine and organic acid content of multiple samples selected randomly throughout a container. A comparison of a dry blend of glucosamine hydrochloride and citric acid, and a GOA comprising the same species is provided in Example 7. The GOA was prepared eleven months prior to homogeneity testing. The dry blend was freshly prepared. The citric acid content of the dry blend varied four times as much as the citric acid content of the GOA. The variation in citric acid measurements in GOA was no greater than that for pure citric acid.

2. Uses of GOA

Embodiments of the GOA are useful for making tablets, especially chewable tablets. Homogeneity is a desirable trait for dry components that are used for making tablets, powdered nutritional supplements, and/or food additives. U.S. Pat. No. 3,619,292 (herein incorporated by reference) describes several methods of making tablets. These basic methods and other methods known in the art can be used to make tablets that contain embodiments of the GOA.

GOA is also desirable because it is believed that the combination of organic acid and glucosamine will allow the glucosamine to have increased bioavailability, similar to the increased calcium bioavailability shown for calcium citrate compounds described in U.S. Pat. No. 4,814,177. Bioavailability can be defined as the relative amount of the dose of a drug or other substance reaching the systemic circulation. One method of testing for bioavailability is to administer a known quantity of a substance to a subject and then test for the amount of that substance which is excreted from the subject's body. Methods of testing for the bioavailiability of glucosamine are well known in the art. For example, Setnikar and Rovati, Arzneimittelforschung 51: 699-725, 2001, describe a method that can be utilized to compare the bioavailability of GOA to glucosamine hydrochloride or glucosamine sulfate.

There are several reasons why homogeneous compositions are advantageous when they are used to formulate pharmaceuticals as described in U.S. Pat. Nos. 6,075,608, 5,054,332, and 5,946,088. For example, homogeneous compositions allow for consistent dosages of active ingredients (such as glucosamine) to be delivered to subjects. Moreover, homogeneous compositions allow for consistent absorption by the body.

### II. EXAMPLES

The following examples serve as illustrations only, and should not serve to limit the scope of protection afforded in the claims. These examples provide methods of making and testing glucosamine organic acid adducts.

### Example 1

### Production of Glucosamine Citrate with Control of Amine-Acid Ratio in Product

The procedure below can be used to prepare glucosamine citrate having various ratios of glucosamine:citrate. The ratios are controlled by the molar ratios in the starting solution, and may range from 10:1 to 1:5 glucosamine:citric acid (G:C). The GOA compositions described below were prepared using starting G:C ratios of 5:1, 3:1, 1:1, 1:2, and 1:5. The examples used partial crystallization rather than complete water removal, demonstrating the ability to use the relative solubilities of glucosamine and the organic acid in controlling the product composition.

More specifically, 1 mole glucosamine hydrochloride (purchased from Pfansteihl Laboratories, Inc, Waukegan, Ill.) was added to 1 L deionized water at room temperature and stirred until there were no visible solids left. 2 moles of citric acid (Cargill, Inc., Eddyville, Iowa) were then added to the dissolved glucosamine solution, hence, creating a 1:2 G:C ratio.

The solution was then placed in a rotary evaporator under a vaccum (25 inches of mercury) at 60° C. until 50-70% of the water evaporated. Crystals resulted from the evaporation step, and these crystals were isolated by vacuum filtration at room temperature. The crystals were then dried in open pans at room temperature.

Using the above procedure, crystals were created that contained the Wt % reflected in Table 1, below.

TABLE 1

| Mole ratio G:C | Wt % | | Wt % Chloride |
|---|---|---|---|
| In starting solution | Glucosamine | Wt % Citric Acid | (balancing anion) |
| 5:1 | 80.9 | 2.1 | 16.1 |
| 3:1 | 73.3 | 9.2 | 14.9 |
| 1:1 | 72.3 | 10.6 | 13.4 |
| 1:2 | 57.4 | 22.9 | 12.6 |
| 1:5 | 31.8 | 58.1 | 5.9 |

### Example 2

### Glucosamine Citrate from Glucosamine, Citric Acid, and NaOH

The procedure below can be used to prepare a glucosamine citrate adduct having various ratios of glucosamine:citrate:NaOH. The ratios are controlled by the molar ratios in the starting solution, and may range from 10:1:0 to 1:5:15 glucosamine:citric acid:NaOH. GOAs were prepared using starting ratios of 10:2:1, 6:2:1, 2:2:1, 1:2:1, and 2:6:3. All tested preparations used 0.5 equivalents of NaOH based on citric acid in the starting solution. At high NaOH ratios (1 citrate:3 NaOH), the solution becomes very dark upon heating, indicating chemical reaction of the amine. Other bases, such as KOH, may also be used. The GOA samples prepared in this example used partial evaporation of water as in Example 1 to illustrate the effects of relative solubilities.

A solution containing glucosamine hydrochloride and citric acid (1:2 G:C) was made as described above. To that solution 1 mole of NaOH was slowly added and mixed thoroughly at room temperature.

Crystals were formed, filtered, and dried using the techniques described above.

Using the above procedure crystals were created that contained the Wt % reflected in Table 2, below.

TABLE 2

| Mole ratio G:C | Wt % Glucosamine | Wt % Citric Acid | Wt % Chloride |
|---|---|---|---|
| 5:1 | 80.4 | 1.9 | 16.2 |
| 3:1 | 79.3 | 3.6 | 16.1 |
| 1:1 | 78.2 | 4.2 | 13.9 |
| 1:2 | 69.2 | 12.3 | 10.5 |
| 1:3 | 56.3 | 13.5 | 10.2 |

### Example 3

### GOA Preparation from Glucosamine Sulfate and Citric Acid

The procedure from Example 1 was used to prepare glucosamine citrate from glucosamine sulfate [(GlcN)₂SO₄.2KCl] (distributed by Anhui Worldbest, Hefei, P.R. China), and citric acid (Cargill, Inc., Eddyville, Iowa). 0.2 mole of glucosamine sulfate was dissolved in deionized water. The citric acid (0.2 mole) was added and dissolved. The solution was evaporated on a rotary evaporator at 60° C. and 25 inches of vacuum until crystals formed. The crystals were isolated by vacuum filtration, and then air-dried at room temperature.

The composition of the GOA thus formed was 65.4 wt % glucosamine, 12.4 wt % chloride, 2.5 wt % K, 3.7 wt % sulfate, and 15.2 wt % citric acid.

### Example 4

### GOA Preparation from Glucosamine Hydrochloride, Trisodium Citrate, and Citric Acid

The procedure in Example 1 was adapted to prepare GOA from glucosamine hydrochloride, trisodium citrate (TSC), and citric acid, anhydrous (CAA). In this example, the trisodium citrate was used in place of the sodium hydroxide from Example 2 to neutralize the glucosamine hydrochloride.

Two 0.2 mole aliquots of glucosamine hydrochloride (Pfanstiel Laboratories, Inc., Waukegan, Ill.) were dissolved in deionized water, then cooled to approximately 5° C. in an ice-water bath. 0.07 mole (0.2 equivalent) of trisodium citrate (Cargill, Inc., Eddyville, Iowa) was added to one aliquot, and then mixed until all solids dissolved. Another 0.13 mole of citric acid (Cargill, Inc., Eddyville, Iowa) was added to the solution, and then mixed until all solids dissolved.

A dry mixture of trisodium citrate and citric acid (0.07 and 0.13 moles, respectively) was added to the second aliquot of glucosamine hydrochloride solution. The solution was mixed until all solids dissolved.

The solutions were transferred to separate rotary evaporator flasks and evaporated at 60° C. and at 25 inches of vacuum until crystals formed. The crystals were separated from the liquors by vacuum filtration, then air-dried at room temperature. The crystals formed exhibited the Wt % composition in Table 3, below.

TABLE 3

| Citrate | Wt % | Wt % | | Wt % |
|---|---|---|---|---|
| Addition | Glucosamine | Citric Acid | Wt % Sodium | Chloride |
| TSC, followed | 70.3 | 11.7 | 1.5 | 13.8 |
| by CA | | | | |
| TSC and CA | 72.9 | 9.4 | 1.1 | 14.2 |

### Example 5

### GOA Production from Glucosamine Hydrochloride and Lactic or Malic Acids

The procedure of Example 1 was used to prepare GOA using lactic or malic acids. Lactic acid (USP grade, Mallinckrodt, Paris, Ky.) was tested at mole ratios of one and two with respect to glucosamine hydrochloride. Malic acid (Parchem Trading, LID, White Plains, N.Y.) was tested at mole ratios of 1, 2 and 3.

Using the procedure in Example 1, crystals were created that contained the Wt % reflected in Table 4, below. In the first column, the letters G, L, and M refer to glucosamine, lactic acid, and malic acid, respectively.

TABLE 4
Characteristics of GOA from Lactic and Malic Acids

| Mole Ratio | Wt % Acid | Wt % Glucosamine | Wt % Chloride |
|---|---|---|---|
| 1G:1L | 1.2 | 81.8 | 15.7 |
| 1G:2L | 3.0 | 79.3 | 15.8 |
| 1G:1M | 6.6 | 78.0 | 15.3 |
| 1G:2M | 12.2 | 72.9 | 14.4 |
| 1G:3M | 8.5 | 75.4 | 15.2 |

The high water solubilities of malic and lactic acids compared to citric acid account for the lower acid contents of the GOAs produced in this example.

### Example 6

### Characterization and Identification of GOA

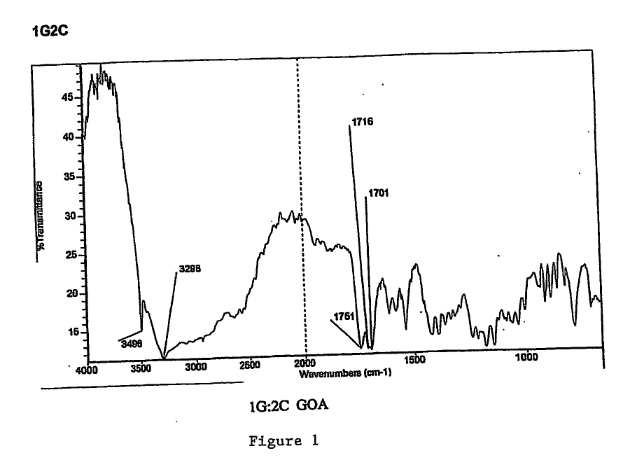

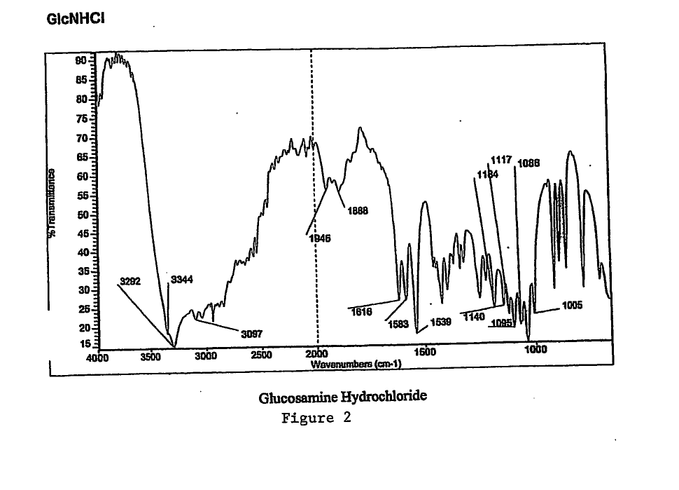

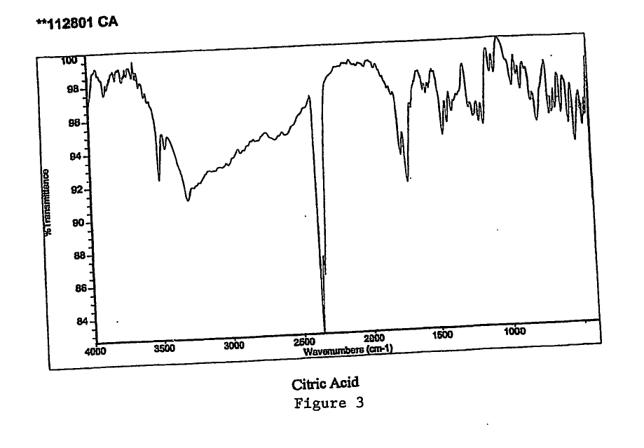

The GOA products made in Examples 1 through 5 can be analyzed for concentration of their components by FTIR (Fourier Transform Infrared) Spectrometry. Using standard FTIR techniques well known in the art, spectra were generated (see USP-NF monograph for glucosamine, published 2002). The spectra shows bands characteristic of both citric acid and glucosamine, as indicated in FIGS. 1, 2, and 3. Proximate analysis to determine the molar ratios in the adduct is accomplished by the methods described below.

Citric acid is determined quantitatively by dissolving a known quantity of glucosamine citrate in deionized water. The solution is filtered through a 0.22μ filter into a HPLC vial. The sample is analyzed by HPLC using a BioRad HPX-87H column (BioRad, City, State) and 0.01 N H₂SO₄ as the mobile phase.

Glucosamine was determined by a total nitrogen measurement using a LECO or Antek nitrogen analyzer according to the manufacturer's instructions. Since the purity of glucosamine is known before preparing the glucosamine-citrate, a total nitrogen value can be applied.

Other inorganic species, including sodium, potassium and sulfate were determined by ICP-AES (inductively coupled plasma-atomic emission spectrometry). Certified standards were used to calibrate the instrument.

Residual chloride is determined using a potentiometric titration. Silver nitrate is the titrant, and a silver indicating electrode monitors the course of the titration.

Moisture was determined using either an oven at 105° C., where the percent change in sample weight after drying is due to moisture or a halogen moisture balance (Mettler or equivalent), which heats the sample while monitoring weight, stopping when weight change ceases.

### Example 7

### Homogeneity of GOA

A sample containing a blend comprising 20% by weight anhydrous citric acid and 80% glucosamine hydrochloride was blended by tumbling for five minutes. The blended sample was poured into a tray. Eight aliquots were collected from different areas of the tray. The aliquots were diluted in 0.01N H₂SO₄, and then analyzed for citric acid by the method described in Example 6. The peak areas were normalized by the aliquot weights, and then averaged. The relative standard deviation for the eight aliquots was 7.7%.

The two components in the blend had similar, but not identical particle size distributions. The natural crystal shapes of these components differ, in that citric acid crystals are needle shaped while glucosamine hydrochloride crystals are bipyramidal.

A sample of GOA from Example 2, which had been stored at room temperature in a flask for approximately eleven months, having 12.3% citric acid was analyzed similarly. Eight aliquots were taken from the sample. Citric acid was determined by HPLC as described above. The peak areas were normalized by the aliquot weights, and then averaged. The relative standard deviation for the eight aliquots was 1.9%.

The analytical precision measured from eight aliquots of pure citric acid was 1.8% relative. The difference in precision between the GOA and citric acid was negligible, demonstrating a high degree of homogeneity. In contrast, the differences in precision between pure citric acid and the dry blend was significant, indicating a low degree of homogeneity.

### Example 8

### Taste of GOA

The GOA composition offers the ability to alter taste profile when compared to glucosamine hydrochloride or glucosamine sulfate. Taste was measured by conducting a “difference from control test” using a panel of 7 individuals. Panelists evaluated samples by comparing glucosamine hydrochloride {(control) (Lot 27126A), CAS No. 66-84-2 (Phanstiehl Laboratories, Inc., Waukegan, Ill.)} to unmarked glucosamine organic acid samples (glucosamine citrate) in various ratios and a blind control at 0.44% weight/weight glucosamine hydrochloride in water and reporting how samples were the same or different. The sensory panelists found very apparent differences in the samples as summarized below in Table 5. Samples were also rated on a 5 point scale for difference from control, 0=no difference, 1=trace, 2=slight, 3=moderate, 4=strong, 5=very strong.

TABLE 5
GOA Sensory Results

| | Numerical Results | |
|---|---|---|
| Sample | (Average) | Taste Results |
| Blind Control | 1 | Trace sweet, bitter |
| 1G:1C | 4 | Strong sour, slight astringent |
| 1G:2C | 5 | Very strong sour, strong astringent |
| 1G:3C | 5 | Very strong ++sour, very strong |
| | | astringent |

All samples were different from the control, effectively reducing the bitterness of glucosamine. “Astringent” is defined as dryness in the mouth. These results indicate that an adduct of glucosamine with an organic acid has desirable taste characteristics when compared to pure glucosamine. Organic acids that are useful for forming the adduct include citric acid, lactic acid, malic acid, and sorbic acid (potassium sorbate).

### Example 9

### Tableting of GOA

GOA has tableting characteristics similar to glucosamine, so existing glucosamine tableting equipment is sufficient for tableting GOA. In order to show this, tablet processing was done manually using a Chemplex Manually Operated Hydraulic Press with a Chemplex Evacuable XRF Sample Pellet Die Assembly. A direct compression method was utilized by adding 1.794 g of glucosamine (Lot 27126A), CAS No. 66-84-2 (Phanstiehl Laboratories, Inc., Waukegan, Ill.) and 1.2 g of binder (Avicel® PH-302 Microcrystalline Cellulose from FMC®) to a 50 mL tube and shaking vigorously for 10 minutes in an automated shaker, then adding 0.006 g of lubricant (magnesium stearate from Mallinckrodt) and again shaking for 3 minutes, and tableting at 5 tons pressure and under <2 cm Hg of vacuum. Tablets were also prepared with this method, but GOA was used instead of glucosamine. All tablets were comparable in regard to release from the die, and there was no noticeable chipping or stress cracking.

Therefore, based on the enhanced flavor of GOA compared to glucosamine, the formation of chewable tablets for those who are unable to swallow large pills or prefer a different delivery system can be achieved.

### Example 10

### Preparation of GOA by Surface Crystallization

Examples 1 through 5 demonstrated GOA preparation where glucosamine and organic acids are in solution, then crystallized as adducts. GOA can be prepared by spraying a solution of one component onto the other component in solid form. The moisture is removed, resulting in GOA where the surfaces of the crystals contain glucosamine and organic acids, but the solid component is never fully dissolved, such that the cores of the particles may contain only a single component.

To prepare GOA in this manner, a rotary tumbler is partially filled with solid glucosamine hydrochloride. The material is tumbled while a fine aerosol consisting of a 20 wt % solution of citric acid in water is sprayed onto the tumbling solids. Heat is added to control evaporation rate, maintaining a moisture level too low to dissolve the glucosamine crystals. Once the desired amount of citric acid is added, the remaining moisture is removed before tumbling is stopped.

The GOA product produced will be homogeneous when tested at typical dosage levels (0.1 gram or more per sample).

1. A glucosamine organic acid adduct comprising glucosamine, and one or more organic acids.

2. The adduct of claim 1, wherein the adduct is substantially homogenous.

3. The adduct of claim 1, further comprising salts of organic acids, moisture, one or more balancing ions, and one or more inorganic salts

4. The adduct of claim 1 in a tablet form.

5. The adduct of claim 1, wherein the molar concentration of glucosamine is greater than or equal to the molar concentration of organic acid.

6. The adduct of claim 1, wherein the molar concentration of glucosamine is less than the molar concentration of organic acid.

7. The adduct of claim 1 wherein the content of the moisture is less than about 20 weight percent.

8. The use of the adduct of claim 1 as a dietary supplement.

9. The adduct of claim 8 wherein the glucosamine and the one or more organic acids in the single compound are in a molar ratio of 5:1.

10. The adduct of claim 8 wherein the moisture content of said composition is less than about 20 weight percent.

11. The use of the adduct of claim 1 as a food ingredient.

12. The composition of claim 11 wherein glucosamine and the one or more organic acids in the single compound are in a molar ratio of 5:1.

13. A process for producing a glucosamine organic acid adduct comprising:
forming a solution comprising an aqueous medium, glucosamine and an organic acid;
removing the aqueous medium; and
collecting the crystals, wherein the crystals comprise the glucosamine organic acid adduct.

14. The process according to claim 13, wherein substantially all of the aqueous medium is removed.

15. The process according to claim 13, further comprising adding a base to the solution prior to removing water from the solution.

16. The process according to claim 13, wherein some or all of the water is replaced by a water miscible solvent.

17. A product created by the process of claim 13.

18. A composition consisting of glucosamine, one or more organic acids or salts thereof, moisture, one or more balancing ions, and one or more inorganic salts.

19. A method of forming a glucosamine organic acid adduct comprising:
tumbling a dry component comprising glucosamine and/or one or more organic acids; and
spraying the dry component with a solution comprising glucosamine, and/or one or more organic acids, wherein a glucosamine organic acid adduct is formed.
